(19) United States
(12) Patent Application Publication
(10) Pub. No.: US 2019/0242890 A1
(43) Pub. Date: Aug. 8, 2019

(54) METHOD FOR ENHANCING THE INCUBATION OF SAMPLES, SPECIMENS AND REAGENTS USING LASERS

(71) Applicant: Jim MANOLIOS, Red Hill (AU)

(72) Inventor: Jim MANOLIOS, Red Hill (AU)

(21) Appl. No.: 16/340,853

(22) PCT Filed: Oct. 11, 2017
(86) PCT No.: PCT/AU2017/000215

§ 371 Date: Apr. 10, 2019

(30) Foreign Application Priority Data
Oct. 11, 2016 (AU) 2016904111

(51) Int. Cl. G01N 33/555; C12M 1/00; C12M 1/02; C12M 1/26

(52) U.S. Cl. CPC G01N 33/555; C12M 33/10; C12M 41/18; C12M 41/14

(57) ABSTRACT

A method for incubating reagents, suspensions and/or biological products to a predetermined temperature for biological testing; the method comprising the steps of: a) providing a selected biological product in a receptacle and which is required to be incubated to a temperature which simulates a natural thermal environment of the biological product; b) applying controlled heating to or near the biological product using at least one laser emitting from a laser source for a time sufficient to reach a target temperature or which simulates a target temperature of the natural thermal environment in which the biological product exists.

### BACKGROUND

The present invention relates to diagnostic methodologies and more specifically relates to a diagnostic method in which a laser is used to enhance the incubation of biological samples and reagents for performing in-vitro diagnostics and particularly in various forms of Blood Group Serology testing. The present invention also relates to a method of serological testing of biological products using a laser to improve the speed of incubation and to enable more accurate simulation of environmental temperatures in which the biological products are normally found thereby enhancing, maintaining and increasing the accuracy of test results. The invention further relates to a method of testing of biological and other samples using a laser or lasers to either create or simulate in vivo environmental temperature conditions in which the samples exist. The invention also relates to a laser incubation method for Monitoring of Biological Products. Furthermore the invention provides a method for specifically targeting the enhancement or disruption of specific antigen/antibody binding sites in order to specifically either enhance binding or modify the binding site in such a way as to prevent binding and thus alter the antigenic properties of a red cell.

### PRIOR ART

A serological blood test is performed to detect and measure the levels of antibodies as a result of exposure to a particular bacteria or virus. When people are exposed to bacteria or viruses (antigens), their body's immune system produces specific antibodies against the organism. Serology is the scientific study of serum and other bodily fluids. In practice, the term usually refers to the diagnostic identification of antibodies in the serum.

Such antibodies are typically formed in response to an infection (against a given microorganism), against other foreign proteins (in response, for example, to a mismatched blood transfusion), or to one's own proteins (in instances of autoimmune disease). Serological tests may be performed for diagnostic purposes; when an infection is suspected, in rheumatic illnesses, and in many other situations, such as checking an individual's blood type. Serology blood tests help to diagnose patients with certain immune deficiencies associated with the lack of antibodies, such as X-linked agammaglobulinemia. Blood in part is a suspension of proteins most of which have antigenic properties whether they be antigens or antibodies. These proteins create antigenic responses in the body when they come into contact with their complementary antigens/antibodies. This process includes the immune response generated by the body. By modifying the short and long chain proteins that form these antigens and antibodies it is possible to disrupt antigen/antibody binding. An example of this is in the use of human blood to create IVIg products for infusion into humans. Due to the vast array of protein fragments in human blood (especially when it is pooled), patients suffer non descript immune reactions after infusion. If the protein fragments could be selectively denatured and or the patient's blood could be modified to prevent these reactions, a significant limitation to the use of such products could be eliminated.

There are several serology techniques that can be used depending on the antibodies being studied. These include: ELISA, agglutination, precipitation, complement-fixation, and fluorescent antibodies. Serologic blood tests look for antibodies in the blood. They can involve a number of laboratory techniques. Different types of serologic tests can diagnose various disease conditions. Serologic tests all have in common that they all focus on proteins made by the immune system. The process for having the test is the same regardless of which technique the laboratory uses during serologic testing.

Antigens are substances that provoke a response from the immune system. They are most often too small to see with the naked eye. They can enter the human body through the mouth, through broken skin, or through the nasal passages. Antigens that commonly affect people include the following: bacteria, fungi, viruses, parasites. The immune system defends against antigens by producing antibodies. These antibodies are particles that attach to the antigens and deactivate them. A serum antigen blood test can identify the type of antibodies and antigens that are in a blood sample and identify the type of infection in that sample. Sometimes the body mistakes its own healthy tissue for outside invaders and produces unnecessary antibodies. This is known as an autoimmune disorder which may be detected by serological testing which detects these antibodies.

Blood Group Serology testing includes several tests that require an incubation phase, generally at 37 degrees Celsius, room temperature or other variations therein. 37 degree incubation is used to mimic the sample conditions in-vivo that are vital in the correct identification of blood group related antibodies and ‘cold agglutinants’. Traditional 37 degree incubation has been achieved using a water bath, hot air incubator or some form of metallic heat transfer device. Each of the aforesaid incubation methods have attendant disadvantages:

Water baths are messy, can contaminate the test devices (usually glass or plastic tubes) and can be a significant source of bacterial contamination which can affect the test and user alike. Hot air incubators can dry out the test/samples, take a long time to bring the sample up to temperature and can cause unwanted aerosols of potentially infectious materials. In response to those disadvantages the industry employed Aluminum blocks but they have the disadvantage that they can take a significant amount of time to bring the sample under incubation to the correct temperature without exceeding the target temperature (usually 37 C) which can be damaging or even negate the benefits of incubation.

Gel tests are known for the determination of immunoglobulin classes and subclasses and complement fractions coating RBCs. These tests simplified serologic characterization of autoantibodies in various autoimmune diseases. It is important to serologically characterize autoantibodies in autoimmune disorders to effectively predict the prognosis and disease outcome. This characterization can be performed effectively with the gel test, which can be introduced in blood centers as a replacement to the conventional tube technique.

Blood group serology testing in Gel Cards in particular can exacerbate the aforesaid disadvantages of the known methods due to the large mass of the plastic cards and gel material versus the biological sample and reagent red cells, typically 40-75 microlitres. Furthermore, in order to best ‘mimic’ the in-vivo conditions from which the antibody samples have been removed, the antibodies and antigens must be incubated or kept at temperature long enough for the binding sites on the antibodies to ‘unfold’ and be in the same shape as they occur in the body. Historically blood group serology has been (and still is) an empirical science. Although the tests are known to work it is not known which sites are being activated, whether there is cross binding during incubation or whether the presence of certain binding sites can mask other weaker sites.

All incubations have 3 distinct thermal segments:
a) the ramp up time whereby the biological material reaches the correct temperature without significant overshoot of target temperature,
b) The time for the proteins now at temperature to unfold and be available for binding; and
c) the binding phase whereby the antibody binding sites have unfolded and are available for binding with the antigens as they would be in the body.

In the Gel test, the laboratory requires Gel Cards (with preloaded Gel, buffer and anti-human globulin (AHG) reagent), reagent red cells (human red cells of known antigenic profile manufactured to be used in this test system), some form of pipette for loading the reagent red cells and sample (patient plasma), some form of incubator as described above, which is capable of incubating the gel card for up to 15 minutes at 37 degrees Celsius and a centrifuge for spinning the Gel cards once they have been incubated.

The centrifugation step is designed to allow the bound and unbound material proteins to come into contact with the anti human globulin, removing any unwanted binding and allowing any true blood group specific binding that has occurred to pass into and be trapped by the Gel matrix. This testing regime can also be performed automatically using a purpose built analyser. However the protocol is the same, load, incubate, spin, read.

Lasers have a wide variety of uses in industry. They can be employed to heat fluids and although many of the uses of lasers are specifically tailored to achieve a particular result in industry, it has not previously been known to employ a laser to more accurately simulate the in vivo environment from which a sample has been taken for serological testing.

In view of the aforesaid disadvantages of the known art there is a need to improve the methodology associated with the incubation of biological samples and reagents for performing in-vitro diagnostics, and particularly in (though not limited to) various forms of Blood Group Serology testing. There is a need to provide an alternative to and overcome the disadvantages of the known methods including; 1. use of messy water bathes which can cause contamination of samples which affect test results and even the user; 2. methods which dry out the test sample and take a long time to bring the sample up to temperature. There is also a need to increase the efficiency and accuracy of Serological testing and particularly the accuracy of various forms of Blood Group Serology testing in order to overcome or at least ameliorate the shortcomings of the prior art methodology.

### Invention

The present invention provides a method of incubation of biological samples and more particularly provides a method of incubation in which a laser is used to incubate biological products and reagents for performing in-vitro diagnostics, and particularly in but not limited to various forms of Blood Group Serology testing. The present invention further provides a method of serological testing of biological products incorporating the use of a laser to improve the speed of incubation and to enable more accurate simulation of environmental temperatures in which the biological products are naturally found leading to high accuracy in test results. The invention further provides a method for use of laser/infrared technology to enhance the incubation of samples and reagents for performing in-vitro diagnostics, specifically but not limited to various forms of Blood Group Serology testing.

The invention is described with particular (but non limiting), reference to the Gel test. In that test, the laboratory requires:

1. Gel Cards (with preloaded Gel, buffer and anti-human globulin (AHG) reagent),
2. reagent red cells (human red cells of known antigenic profile manufactured to be used in this test system);
3. a form of pipette for loading the reagent red cells and sample (patient plasma),
4. some form of incubator as described above, which is capable of incubating the gel card for up to 15 minutes at 37 degrees Celsius; and
5. a centrifuge for spinning the Gel cards once they have been incubated.

The centrifugation step is designed to allow the bound and unbound material proteins to come into contact with the anti-human globulin, removing any unwanted binding and allowing any true blood group specific binding that has occurred to pass into and be trapped by the Gel matrix. This testing regime can also be performed automatically using a purpose built analyser. However the protocol is the same, load, incubate, spin and read.

In one broad form the present invention comprises: a method for incubating biological products during biological testing; the method comprising the steps of:

taking a selected biological product which is required to be incubated; and
applying controlled heating of the biological product using a laser to a target temperature which simulates a target temperature of the normal environment in which the biological product exists.

According to a preferred embodiment the method includes the further step of aiming the laser directly at the biological product.

In another broad form the present invention comprises:

a method for simulating incubation of biological products during biological testing; the method comprising the steps of:
a) taking a selected biological product;
b) directing at least one laser at the biological product and acting on the biological product to simulate an effect of reaching a predetermined target temperature induced in the biological product,

According to one embodiment the laser is used for the purpose of directly heating the biological sample to the target temperature such that the target temperature is reached in an accelerated time in comparison to conventional incubation. In an alternative embodiment the target temperature is simulated and by modulating the laser wavelength, binding sites on the red cell surface can be preferentially switched on and off. Laser/IR technology is capable of accelerating heating but it is also capable of modulating the laser wavelength binding sites on the red cell surface for the preferential switching on and off.

According to one aspect of the invention, the use of a laser allows an operator to selectively heat the biological sample, or to identify the laser wavelength capable of modulating the red cell antigen binding sites. Thus, the accelerated incubation is achieved directly by laser heating or by achieving the effect of heating by using a laser to simulate a target temperature of the normal environment in which the biological product under test exists. It is therefore possible to simulate required incubation temperature without actually heating the biological sample.

Preferably the laser used is in the green, red or infrared colour spectrums. According to a preferred embodiment the biological products are samples and reagents including a plasma/reagent red cell suspension plasma.

According to one embodiment the method steps are applied in indirect anti-globulin testing (IAT) and in other tests requiring 37 degree Celsius incubation or which require the simulated effect of 37 degree incubation.

In another broad from the present invention comprises: a method of serological testing comprising the steps of:

a) taking a gel card including a gel and having AHG in a gel buffer and which is suitable for indirect anti-globulin testing (IAT) and other tests requiring 37 degree Celsius incubation;
b) using a laser to contact a plasma/reagent red cell suspension via an opening at the top of the Gel Card, so that heating of the Gel Card is avoided;
c) allowing the laser to bring the suspension to 37 degrees;
d) incubating the suspension.

In another broad form the present invention comprises;

a gel card for use in biological testing and having an opening adapted to enable incubation of a biological sample using a laser beam to contact a plasma/reagent red cell suspension via the opening in the Gel Card; the opening in the card arranged so that heating of the Gel Card is avoided in favour of heating the contents of the gel card via the laser beam. The sample may also be heated using a laser through the plastics wall of the test chamber.

Preferably, the opening is located at the top of the Gel card. According to one embodiment, the laser accelerates incubation thereby effectively eliminating a first phase of incubation and greatly enhancing and accelerating the second phase leading to rapid physiological conditions and a much faster incubation.

According to a preferred embodiment there is provided a series of refracted beams from a laser emitter for each well of the gel card. In a further embodiment, the incubation equipment can be built into the centrifuge. This saves significant time, improves productivity and makes automated systems faster and easier to function. Laser incubation provides advantages over the prior art and removes all the limitations of the current available systems. This allows the development of faster, more sensitive and easier to perform testing systems in blood group serology.

In another broad form of the method aspect, the present invention comprises: a method of serological testing using a gel car having an opening therein; the method comprising the steps of;

a) taking the gel card including a gel and having AHG in a gel buffer and which is suitable for indirect anti-globulin testing (IAT) and other tests requiring 37 degree Celsius incubation;
b) using a laser to contact a plasma/reagent red cell suspension directly through the wall of the container or via an opening at the top of the Gel Card, so that heating of the Gel Card is avoided;
c) allowing the laser to bring the suspension to 37 degrees, such that the biological material reaches the target temperature on contact with the laser.

These together with other objects of the invention, along with the various features of novelty which characterize the invention, are pointed out with particularity in the claims annexed to and forming a part of this disclosure. For a better understanding of the invention, its operating advantages and the specific objects attained by its uses, reference should be had to the accompanying illustrations and descriptive matter in which there is illustrated various including preferred embodiments of the invention.

The present invention provides an alternative to the known prior art and the shortcomings identified. The foregoing and other objects and advantages will appear from the description to follow. In the description reference is made to the accompanying representations, which forms a part hereof, and in which is shown by way of illustration specific embodiments in which the invention may be practiced. These embodiments will be described in sufficient detail to enable those skilled in the art to practice the invention, and it is to be understood that other embodiments may be utilized and that structural changes may be made without departing from the scope of the invention. In the accompanying illustrations, like reference characters designate the same or similar parts throughout the several views. The following detailed description is, therefore, not to be taken in a limiting sense, and the scope of the present invention is best defined by the appended claims.

### BRIEF DESCRIPTION

The invention will be better understood and objects other than those set forth above will become apparent when consideration is given to the following detailed description thereof. Such description will now be described in more detail according to a preferred but non-limiting embodiments and with reference to the accompanying illustrations; wherein

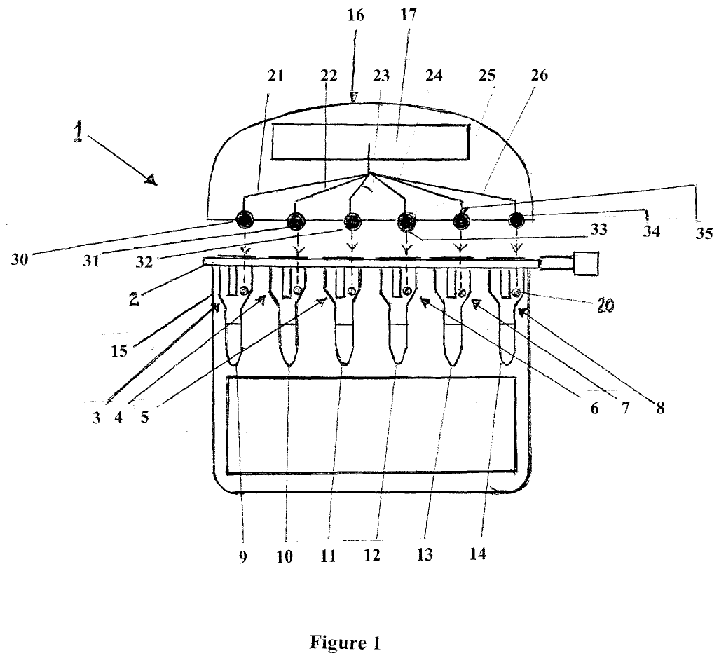

FIG. 1 shows a schematic arrangement of a laser incubation system according to one embodiment.

### DETAILED DESCRIPTION

The present invention will now be described in more detail according to a preferred embodiment but non limiting embodiment and with reference to FIG. 1. The examples referred to herein are illustrative and are not to be regarded as limiting the scope of the invention. While various embodiments of the invention have been described herein, it will be appreciated that these are capable of modification, and therefore the disclosures herein are not to be construed as limiting of the precise details set forth, but to avail such changes and alterations as fall within the purview of the description.

Historically blood group serology is an empirical science, as it is not known which sites are being activated during tests, if there is cross binding during incubation or whether the presence of certain binding sites can mask other weaker sites.

The invention is described herein with reference to Gel Cards for the purpose of illustration but it will be appreciated by persons skilled in the art that the invention has applications apart from blood group serology testing in all its forms and testing protocols and it is contemplated that the method is applicable not only to Gel Cards but to other testing methods in which incubation is used to incubate biological samples at in-vivo temperatures or in which the effects of incubation are simulated. An example of an application in an area other than serological testing is testing new drugs or other materials which are designed to be used in-vivo.

Referring to FIG. 1 there is shown a schematic layout of a laser incubation system 1 according to one embodiment of the invention. System 1 shows a gel card holding plate 2 which receives and retains a plurality of gel cards 3, 4, 5, 6, 7 and 8. Each gel card includes a plurality of vials 9, 10, 11, 12, 13 and 14 which in use are each charged with a reagent, biological specimen, blood product or the like. Holding plate 2 is mounted in a centrifuge 15 and is free to spin under the action of a motor incorporated in the centrifuge 15. This in turn allows the gel cards to spin for separation of specimens as required. The gel card according to one embodiment includes reagent red cells and plasma solution. Incorporated in a lid 16 the centrifuge 15 is a source of laser light 17 from which emanates at least one laser light. In the embodiment shown six laser streams 21, 22, 23, 24, 25, 26 transmit via LED's 30, 31, 32, 33, 34 and 35.

Although six are shown, it will be appreciated that the number of LED laser lights can be changed as required to less than or more than six. The laser lights are transmitted to each of the reagent/biological specimen vials via the LED's. Included in each vial are reagent red cells and plasma suspension 20. In this example, each laser light selects the red plasma cells and the blue green laser preferentially selects the red cells and heats them in suspension while avoiding heating the suspension. The blue/green laser is absorbed by red cells rather than the plasma or other components/constituents. This induces accelerated heating of the biological sample and reduces processing time.

Use of a specific spectrum laser allows incubation of a surface only of a red cell. Also contemplated is the use of an inert dye which will incubate the entire suspension. Thus using the laser heating the operator may select options for heating such as direct heating of the biological sample, indirect heating of the sample by heating the suspension or heating of a surface of a red cell. Although use of a wide range of laser wavelengths is contemplated, a 980 nm laser range is one feasible choice. Also variations in wavelengths and time (duration of heating) may be selected according to the application of the laser heating methodology. There is a relationship between laser wavelengths and time each typified by accelerated direct or indirect heating of vial contents. In the embodiment of FIG. 1 there is provided one laser light per gel card vial. Thus it will be appreciated that incubation of multiple gel cards and multiple vials can be conducted at the same time in one centrifuge.

According to a preferred embodiment, a green, red or infrared laser is used for incubating samples and reagent red cells in performing Gel card based indirect anti-globulin testing (IAT) and other tests requiring 37 degree Celsius incubation. It is known that a laser in the green colour spectrum can heat fluids to approximately 37 degrees Celsius without damaging proteins in the solution.

The present invention allows the laser to contact the plasma/reagent red cell suspension directly through the plastic wall of the container or via an opening at the top of the Gel Card. The laser contacts the biological material specifically thereby avoiding heating the plastic card (the correct laser wavelength does not heat the plastic it passes through) Gel or AHG in the Gel buffer. The action of the laser brings the material to 37 degrees quickly and efficiently enabling a very fast incubation phase.

As a result of this, the use of laser incubation virtually eliminates the first phase of incubation and greatly enhances and accelerates the second phase leading to rapid physiological conditions and a much faster incubation. Furthermore with one laser source per well, the incubation equipment according to one embodiment, can be built into a centrifuge saving a significant amount of time, improving productivity and making automated systems faster and easier to function. Laser incubation potentially overcomes all of the limitations of the current available systems and allows the development of faster, more sensitive and easier to perform testing systems in blood group serology.

The laser incubation system can be adapted to other forms of biological testing but is particularly suited to blood group serology testing in all its forms and testing protocols, including but not limited to Gel card testing. It is contemplated that the laser incubation can be used wherever there is a need to incubate biological samples at in-vivo temperatures. This can be used in testing new drugs or other materials which are designed to be used in-vivo.

It is contemplated that not only can laser/IR technology accelerate heating, but also it is anticipated that by modulating the laser wavelength binding sites on the red cell surface can be preferentially switched on and off. It is further contemplated that the laser can be used to heat or alternatively it may not be necessary to heat the serum and reagent red cells to 37 degrees but rather identify the laser wavelength capable of modulated the red cell antigen binding sites.

The invention is applicable to laser/IR for incubating samples and reagent red cells in performing Gel card based indirect anti-globulin testing (IAT) and other tests requiring 37 degree Celsius incubation. A laser in the green color spectrum for example, excites Haemoglobin in blood and thus heats blood to approximately 37 degrees Celsius without damaging proteins in the solution. Furthermore the invention can be used to enhance specific binding sites on the red cell surface thus preferentially enhancing or preventing antigen binding. This process can also be used to alter the antigenic properties of red cells thus ‘converting’ bold group O positive bold to O negative blood. Furthermore this technique could be used to alter the antigenic state of human blood in vivo via a recirculating pump mechanism.

The present invention allows the laser to contact the plasma/reagent red cell suspension. This may according to one embodiment be via an opening in the Gel Card. This allows contact with the biological material specifically thus not heating the plastic card, Gel or AHG in the Gel buffer. The laser enables a very fast incubation phase. The use of laser incubation eliminates ramp up time for incubation and greatly enhances and accelerates the second phase of protein unfold, leading to rapid physiological conditions and a much faster incubation.

With a link established between wavelength and antigen binding site activation, laser incubation may not require 37 degree incubation but rather a sequential variation in the laser wavelength to selectively activate binding sites and initiate binding in the sample/reagent red cell combination. This can eliminate the need for matched reagent red cell sets as is currently the case, replaced instead with a mixture of cells that contain all known antigen binding sites. Laser incubation potentially addresses all the limitations of the current available systems and will allow the development of faster, more sensitive and easier to perform testing systems in blood group serology.

Apart from blood group serology testing in all its forms and testing protocols i.e. not just Gel Card but all methods, laser incubation could be used wherever there is a need to incubate biological samples in order to activate an antigen-antibody reaction at in-vivo temperatures. This could be used in sandwich Elisa methods or other test methods that require multi-step incubation phases. Other applications include testing new drugs or other materials which are designed to be used in-vivo.

It will be recognised by persons skilled in the art that numerous variations and modification may be made to the invention broadly described herein without departing from the overall spirit and scope of the invention.

The claims defining the invention are as follows:

1. A method for incubating reagents, suspensions and/or biological products to a predetermined temperature for biological testing; the method comprising the steps of:
a) providing a selected biological product in a receptacle and which is required to be incubated to a temperature which simulates a natural thermal environment of the biological product;
b) applying controlled heating to or near the biological product using at least one laser emitting from a laser source for a time sufficient to reach a target temperature or which simulates a target temperature of the natural thermal environment in which the biological product exists.

2. A method according to claim 1 comprising the further step of selecting a time duration for application of laser heating to the biological products.

3. A method according to claim 2 comprising the further step after heating using the at least one laser, of spinning the products in a centrifuge.

4. A method according to claim 3 comprising the further step of aiming the at least one laser directly at the products in suspension.

5. A method according to claim 3 wherein the at least one laser is aimed at a biological specimen under test.

6. A method according to claim 3 comprising the further step of aiming the at least one laser at the reagent to indirectly heat the biological specimen.

7. A method according to claim 3 wherein one laser light is provided for each one of a plurality of vials containing said products.

8. A method according to claim 7 wherein, each laser light selects red plasma cells.

9. A method according to claim 8 wherein there is provided a blue green laser to preferentially select the red plasma cells and heats them in suspension while avoiding heating the suspension.

10. A method according to claim 9 wherein the laser is aimed to induce accelerated heating of the biological sample to reduces specimen processing time.

11. A method according to claim 10 wherein use of a specific selected laser wavelength spectrum allows incubation of a surface only of a red cell.

12. A method according to claim 11 wherein a selected green, red or infrared laser is used for incubating samples and reagent red cells in performing Gel card based indirect anti-globulin testing (IAT) and other tests requiring 37 degree Celsius incubation.

13. A method for incubation of biological products during biological testing; the method comprising the steps of:
a) taking at least one biological product and placing the product is a reagent vial;
b) directing at least one laser from a laser source at or near the biological product to heat the biological product to a predetermined temperature to simulate a natural environment temperature in which the biological product subsists.

14. A method according to claim 13 comprising the further step of directly heating the biological sample using said laser to accelerate elevation to a target temperature.

15. A method according to claim 14 comprising the further step of modulating wavelength of each said at least one laser to preferentially switch on or switch off binding sites on the surface of a red cell.

16. A method according to claim 15 comprising the further step of using the laser to select a temperature to directly heat the biological sample.

17. A method according to claim 16 comprising the further step of identifying a laser wavelength capable of modulating red cell antigen binding sites.

18. A method according to claim 17 comprising the further step of using the laser to select a temperature to simulate a temperature of an environment in which the biological sample exists without directly heating the biological sample.

19. A method according to claim 18 comprising the further step of prior to incubation, selecting a laser from the green, red or infrared colour spectrums.

20. A method according to claim 19 wherein the biological product comprises a plasma/reagent red cell suspension.

21. A method according to claim 20 wherein the method steps are applied in indirect anti-globulin testing (IAT).

22. A method according to claim 21 wherein the method steps are applied in tests on samples requiring 37 degree Celsius incubation.

23. A method according to claim 22 wherein the method steps are applied in tests on samples which require simulated 37 degree Celsius incubation.

24. A method of serological testing comprising the steps of:
a) taking a gel card including a gel and having AHG in a gel buffer and which is suitable for indirect anti-globulin testing (IAT) and tests requiring 37 degree Celsius incubation;
b) using a laser to contact a plasma/reagent red cell suspension via an opening at the top of the Gel Card, so that heating of the Gel Card is avoided;
c) allowing the laser to bring the suspension to 37 degrees;
d) incubating the suspension.

25. A method according to claim 24 comprising the further step of providing a series of refracted beams from a laser emitter for each well of the gel card.

26. A method according to claim 25 comprising the further step of providing laser incubation equipment in or on a centrifuge.

27. A method of serological testing using a gel card having an opening therein; the method comprising the steps of;
a) taking the gel card including a gel and having AHG in a gel buffer and which is suitable for indirect anti-globulin testing (IAT) and other tests requiring 37 degree Celsius incubation;
b) using a laser to contact a plasma/reagent red cell suspension directly through the wall of a vial or via an opening at the top of the Gel Card, so that heating of the Gel Card is avoided;
c) allowing the laser to bring the suspension to 37 degrees, such that the biological material reaches the target temperature on contact with the laser.

28. A gel card for use in biological testing and having an opening adapted to enable incubation therein of a biological sample using at least one laser beam to contact a plasma/reagent red cell suspension via the opening in the Gel Card; the opening in the card arranged so that heating of the Gel Card is avoided in favour of heating the contents of the gel card by the at least one laser beam.

29. A gel card according to claim 28 wherein the opening is located at the top of the Gel card.

30. A gel card according to claim 29 wherein contents of the gel card can be heated through a plastics wall of a test chamber in the gel card.

31. A gel card according to claim 30 incorporated in a centrifuge including a source of laser beams for heating the contents of the gel card.
